(19) United States
(12) Patent Application Publication
(10) Pub. No.: US 2010/0172889 A1
(43) Pub. Date: Jul. 8, 2010

(54) DEGRADABLE BIOMOLECULE COMPOSITIONS

(76) Inventors: Jeffrey M. Catchmark, Bellefonte, PA (US); Burkhard Fugmann, Ratingen (DE); Yang Hu, State College, PA (US)

Correspondence Address: FISH & RICHARDSON P.C., PO BOX 1022, MINNEAPOLIS, MN, 55440-1022, US

(21) Appl. No.: 12/631,447

(22) Filed: Dec. 4, 2009

Related U.S. Application Data
(60) Provisional application No. 61/200,980, filed on Dec. 5, 2008.
(60) Provisional application No. 61/201,002, filed on Dec. 5, 2008.

(51) Int. Cl. A61K 38/43; C12N 9/00; C12N 9/42; A01N 25/00; A01P 1/00; A61P 43/00

(52) U.S. Cl. 424/94.1; 435/183; 435/209

(57) ABSTRACT

This document provides methods and materials related to degradable biomolecule compositions. For example, methods and materials related to compositions having one or more biomolecules and one or more biomolecule degrading enzymes having activity to degrade the one or more biomolecules of the composition are provided. In some cases, the degradable biomolecule compositions provided herein can be used as wound dressings to facilitate wound healing, as tissue scaffolds or tissue matrices to promote tissue growth or tissue regeneration, as bulking agents to provide bulk to tissue in a temporary manner, and as non-medical devices to provide compositions that are degradable.

### CROSS-REFERENCE TO RELATED APPLICATIONS

This application claims the benefit of U.S. Provisional Application Ser. No. 61/200,980, filed Dec. 5, 2008 and U.S. Provisional Application Ser. No. 61/201,002, filed Dec. 5, 2008. The disclosures of the prior applications are considered part of (and are incorporated by reference in) the disclosure of this application.

### BACKGROUND

1. Technical Field

This document provides methods and materials related to degradable biomolecule compositions. For example, this document provides methods and materials related to compositions having one or more biomolecules and one or more biomolecule degrading enzymes having activity to degrade the one or more biomolecules of the composition.

2. Background Information

Enzymes capable of degrading biological materials have evolved to degrade particular substrates and to have activity under a variety of environmental conditions such as high or low temperature, acidic or alkaline pH, and various levels of salinity. For example, plants, some bacteria, fungi, protozoa, and ascidians synthesize cellulose and need to be able to degrade or modify the polysaccharaide during growth and development. Cellulases are enzymes that, in some cases, can hydrolyze and degrade the β-1,4-D-glucan linkages of cellulose into products such as glucose, cellobiose, and cellooligosaccharides. Cellulases can be produced by a number of microorganisms.

### SUMMARY

This document provides methods and materials related to degradable biomolecule compositions. For example, this document provides degradable biomolecule compositions having one or more biomolecules and one or more biomolecule degrading enzymes having the ability to degrade the one or more biomolecules of the composition. As described herein, the degradable biomolecule compositions provided herein can be used as wound dressings to facilitate wound healing, as tissue scaffolds or tissue matrices to promote tissue growth or tissue regeneration, as bulking agents to provide bulk to tissue in a temporary manner, or as non-medical devices to provide compositions that are degradable. In addition, the degradable biomolecule compositions provided herein can be engineered to remain stable prior to use and to have a particular degree and speed of biomolecule degradation upon use. For example, a degradable biomolecule composition provided herein can be engineered to degrade completely within seven days of use (e.g., application to a human's skin or implantation into a living body). Having the ability to prevent degradation of the biomolecules of a composition provided herein before its intended use can allow an end user (e.g., a medical practitioner such as a nurse) to store the composition in a stable manner for extended periods. In addition, having the ability to control the degree and speed of biomolecule degradation of the compositions provided herein can allow medical practitioners to select an appropriate composition for a particular medical application or treatment. For example, a doctor can select a tissue matrix that completely degrades within three to four weeks when treating a fast healing tissue and can select a tissue matrix that completely degrades within three to four months when treating a slow healing tissue.

In general, one aspect of this document features an engineered composition comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features the use of a composition as a medical device. The composition (e.g., an engineered composition) can comprise, or consist essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide. The medical device can be a wound dressing or tissue scaffold.

In another aspect, this document features the use of a composition in the manufacture of a medical device. The composition (e.g., an engineered composition) can comprise, or consist essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide. The medical device can be a wound dressing or tissue scaffold.

In another aspect, this document features the use of a composition in the manufacture of a wound dressing or tissue scaffold to treat an injury. The composition (e.g., an engineered composition) can comprise, or consist essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features a method of manufacturing a composition (e.g., an engineered composition). The composition can comprise, or consist essentially of, one or more biomolecules and one or more biomolecule degrading enzymes. The method comprises, or consists essentially of, lyophilizing the one or more biomolecules and the one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination.

In another aspect, this document features a medical device comprising, or consisting essentially of, a composition. In some cases, the composition can be a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. In some cases, the composition can be a composition (e.g., an engineered composition) comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide. The medical device can be a wound dressing or tissue scaffold. The medical device can comprise, or consist essentially of, a first layer comprising the one or more biomolecules and a second layer adjoining the first layer and comprising the one or more biomolecule degrading enzymes. The medical device can comprise, or consist essentially of, a first layer comprising the one or more biomolecules, a second bioabsorbable layer adjoining the first layer, and a third layer adjoining the second layer and comprising the one or more biomolecule degrading enzymes. The second layer can lack the one or more biomolecules of the first layer.

In another aspect, this document features a method for rehydrating a medical device comprising one or more lyophilized biomolecules and one or more lyophilized biomolecule degrading enzymes. The method comprises, or consists essentially of, contacting the medical device with an aqueous solution or water prior to being placed into contact with biological cells or tissue.

In another aspect, this document features a method for rehydrating a medical device comprising one or more lyophilized biomolecules and one or more lyophilized biomolecule degrading enzymes. The method comprises, or consists essentially of, contacting the medical device with an aqueous solution or water outside a patient to be treated.

In another aspect, this document features a method of treating a patient comprising administering to the patient a medical device comprising, or consisting essentially of, a composition. In some cases, the composition can be a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. In some cases, the composition can be a composition (e.g., an engineered composition) comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide. The medical device can be a wound dressing or tissue scaffold. The medical device can comprise, or consist essentially of, a first layer comprising the one or more biomolecules and a second layer adjoining the first layer and comprising the one or more biomolecule degrading enzymes. The medical device can comprise, or consist essentially of, a first layer comprising the one or more biomolecules, a second bioabsorbable layer adjoining the first layer, and a third layer adjoining the second layer and comprising the one or more biomolecule degrading enzymes. The second layer can lack the one or more biomolecules of the first layer.

In another aspect, this document features a drug delivery device comprising, or consisting essentially of, a drug and a composition. In some cases, the composition can be a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. In some cases, the composition can be a composition (e.g., an engineered composition) comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features the use of a composition for agriculture materials, filter materials, insulating materials, packaging materials, food, or dietary supplements. In some cases, the composition can be a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. In some cases, the composition can be a composition (e.g., an engineered composition) comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features an agriculture material, filter material, insulating material, packaging material, food, or dietary supplement comprising, or consisting essentially of, a composition. In some cases, the composition can be a composition produced by a method comprising, or consisting essentially of, lyophilizing one or more biomolecules and one or more biomolecule degrading enzymes. The method can comprise, or consist essentially of, (a) lyophilizing the one or more biomolecules to form lyophylized biomolecules, (b) applying the one or more biomolecule degrading enzymes to the lyophylized biomolecules to form a combination, and (c) lyophilizing of the combination. In some cases, the composition can be a composition (e.g., an engineered composition) comprising, or consisting essentially of, one or more biomolecules and one or more biomolecule degrading enzymes. The one or more biomolecules can be rehydratable biomolecules. The one or more biomolecule degrading enzymes can be rehydratable biomolecule degrading enzymes. The composition can be bioabsorbable. The composition can be biodegradable. The one or more biomolecules can be selected from the group consisting of keratin, collagen, elastin, starch, cellulose, chitosan, and chitin. The one or more biomolecule degrading enzymes can be capable of degrading the one or more biomolecules. The one or more biomolecules can form a 2- or 3-dimensional matrix. The matrix can be porous. The one or more biomolecule degrading enzymes can be evenly distributed within the matrix. The one or more biomolecules can be structural polysaccharides, and wherein the one or more biomolecule degrading enzymes are structural polysaccharide degrading enzymes. The structural polysaccharides can be selected from the group consisting of β-linked 1,4 polysaccharides, β-linked 1,3 polysaccharides, α-linked 1,4 polysaccharides, and α-linked 1,3 polysaccharides. The structural polysaccharide degrading enzymes can be selected from the group consisting of β-linked 1,4 polysaccharide degrading enzymes, β-linked 1,3 polysaccharide degrading enzymes, α-linked 1,4 polysaccharide degrading enzymes, and α-linked 1,3 polysaccharide degrading enzymes. The composition can comprise cellulose and one or more cellulose degrading enzymes. The cellulose can be nanodimensional. The cellulose can be microbially-derived cellulose. The microbially-derived cellulose can be acetobacter-derived cellulose or glucanobacter-derived cellulose. The cellulose degrading enzymes can be selected from the group consisting of endoglucanases, exoglucanases, and β-glucosidases. The cellulose degrading enzymes can be selected from the group consisting of acid cellulases, hybrid cellulases, neutral cellulases, and alkaline cellulases. The composition can further comprise at least one compound selected from the group consisting of biocides, pharmaceuticals, growth promoting agents, biomolecule degrading enzyme inhibitors, protease inhibitors, and pH level controlling agents. The one or more biomolecules can be lyophilized biomolecules. The one or more biomolecule degrading enzymes can be lyophilized biomolecule degrading enzymes. The one or more biomolecules and the one or more biomolecule degrading enzymes can be made rehydratable by a lyophylization process. The composition can comprise one or more chemicals capable of adjusting the pH of an environment contacted with the composition. The one or more chemicals can be selected from the group consisting of hydrochloric acid, sodium acetate, phosphoric acid, acetic acid, citric acid, lactic acid, and sodium bicarbonate. The one or more biomolecules can be structural proteins, and the one or more biomolecule degrading enzymes are structural protein degrading enzymes. The structural proteins can be selected from the group consisting of keratin, collagen, and elastin. The composition can further comprise polyhexamethylene biguanide.

In another aspect, this document features a meat substitute comprising, or consisting essentially of, a composition. The composition comprises, or consists essentially of, one or more tissues grown using the composition.

In another aspect, this document features a method of treating a subject in need thereof with a medical device comprising one or more lyophilized biomolecules and one or more lyophilized biomolecule degrading enzymes. The method comprises, or consists essentially of, contacting the medical device with an aqueous solution or water prior to contacting the medical device with biological cells or tissue of the subject. The medical device can be brought into contact with the aqueous solution or water outside the subject to be treated.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description and from the claims.

### DESCRIPTION OF DRAWINGS

FIG. 1 is a schematic representation of one example of a wound care device having a first layer that includes a biomolecule (e.g., a cellulose material) and a second layer that includes a biomolecule degrading enzyme (e.g., a cellulose degrading enzyme).

FIG. 2 is a schematic representation of one example of a wound care device having a first layer that includes a biomolecule (e.g., a cellulose material), a second layer that includes a synthetic bioabsorbable material, and a third layer that includes a biomolecule degrading enzyme (e.g., a cellulose degrading enzyme).

### DETAILED DESCRIPTION

This document provides methods and materials related to degradable biomolecule compositions. For example, this document provides degradable biomolecule compositions as well as methods of using such degradable biomolecule compositions. In general, a degradable biomolecule composition provided herein can be designed to be stable such that the biomolecules of the composition exhibit little or no degradation until the biomolecule degrading enzymes of the composition are placed in contact with the biomolecules and/or are activated. Once placed in contact and/or activated, the biomolecule degrading enzymes of the composition can proceed to degrade the biomolecules of the composition. The degradable biomolecule compositions can be designed as described herein to control the degree and speed of biomolecule degradation by the activated biomolecule degrading enzymes.

The degradable biomolecule compositions provided herein can be engineered compositions. The term “engineered” as used herein with respect to a composition refers to a composition designed, developed, constructed, and/or made by man. For example, a degradable biomolecule composition provided herein can be an engineered composition that was constructed by man.

The degradable biomolecule compositions provided herein can have one or more biomolecules (e.g., isolated biomolecules) and one or more biomolecule degrading enzymes (e.g., isolated biomolecule degrading enzymes) having the ability to degrade the one or more biomolecules of the composition. As used herein, the term “biomolecule” refers to any organic molecule such as a polypeptide, polysaccharide, or nucleic acid, or a derivative thereof, that is produced by a living organism. The term “biomolecule” also refers to engineered and/or non-naturally occurring analogs of naturally occurring organic molecules. A degradable biomolecule composition provided herein can be designed to include any appropriate biomolecule. Examples of biomolecules that can be used to make a degradable biomolecule compositions provided herein include, without limitation, polysaccharides, polypeptides, peptides, oligosaccharides, nucleic acids such as oligonucleotides and/or polynucleotides, and combinations thereof.

The term “isolated” as used herein with respect to biomolecules or biomolecule degrading enzymes refers to biomolecules or biomolecule degrading enzymes that have been separated from at least one cellular component with which they are naturally accompanied. In some cases, an isolated biomolecule or biomolecule degrading enzyme can be substantially pure. Typically, a biomolecule or biomolecule degrading enzyme provided herein is substantially pure when it is at least 50 percent (e.g., 60, 65, 70, 75, 80, 90, 95, or 99 percent), by weight, free from proteins and naturally-occurring organic molecules with which it is naturally associated. In general, a substantially pure biomolecule or biomolecule degrading enzyme will yield a single major band on a non-reducing polyacrylamide or agarose gel.

In some cases, a degradable biomolecule composition provided herein can be designed to include one or more polysaccharides (e.g., homopolysaccharides, heteropolysaccharides, or combinations thereof). The polysaccharides of a degradable biomolecule composition provided herein can be complex carbohydrates made up of monosaccharides joined together by glycosidic bonds. Examples of polysaccharides that can be used to make a degradable biomolecule composition provided herein include, without limitation, storage polysaccharides such as starch and glycogen as well as structural polysaccharides such as cellulose and chitin. In some cases, polysaccharides such as β-linked 1,4 polysaccharides, β-linked 1,3 polysaccharides, α-linked 1,4 polysaccharides, α-linked 1,3 polysaccharides, or combinations thereof can be used to make a degradable biomolecule composition provided herein.

In some cases, a degradable biomolecule composition provided herein can be designed to include cellulose and/or chitin. In such cases, the cellulose and/or chitin can be formulated such that it is or remains biocompatible. In some cases, cellulose and/or chitin can be produced to have a desired degree of porosity and/or a desired shape. For example, different types of cellulose fibers can be designed and incorporated into a degradable biomolecule composition provided herein. In some cases, nanodimensional or nanocrystalline cellulose (e.g., nanodimensional cellulose fibrils where the diameter of the fibrils ranges from about 2 to 100 nm) can be used to make a degradable biomolecule composition provided herein.

In some cases, a degradable biomolecule composition provided herein can include one or more cellulose derivatives. Examples of cellulose derivatives include, without limitation, carboxylmethyl cellulose, cellulose acetate, cellulose diacetate, cellulose triacetate, and other cellulose materials with altered chemistry. Any appropriate method can be used to obtain a cellulose derivative. For example, carboxylmethyl cellulose can be produced by heating cellulose with a caustic solution (e.g., a solution of sodium hydroxide) and treating it with methyl chloride. In a substitution reaction that follows, the hydroxyl residues (—OH functional groups) can be replaced by methoxide (—OCH₃ groups). The production of cellulose acetate can involve the following steps. Purified cellulose can be reacted with acetic acid and acetic anhydride in the presence of sulfuric acid. It can then be put through a controlled, partial hydrolysis to remove the sulfate and a sufficient number of acetate groups to give a product with desired properties. The anhydroglucose unit can be the fundamental repeating structure of cellulose and can have three hydroxyl groups that can react to form acetate esters. A common form of cellulose acetate fiber can have an acetate group on about two of every three hydroxyls. Such a cellulose diacetate can be referred to as a secondary acetate, or simply as “acetate.” After it is formed, cellulose acetate can be dissolved in acetone into a viscous resin for extrusion through spinnerets, which can resemble a shower head. As the filaments emerge, the solvent can be evaporated in warm air via dry spinning, producing fine cellulose acetate fibers.

In some cases, a degradable biomolecule composition provided herein can be designed to include microbially-derived cellulose. For example, microbial cellulose derived from acetobacter (e.g., Acetobacter xylinum, Acetobacter acetigenus) and/or gluconobacter (e.g., Gluconobacter oxydans) can be used to make a degradable biomolecule composition provided herein. In general, bacteria such as Acetobacter xylinum can extrude glucan chains from pores into its growth medium. The glucan chains can aggregate into microfibrils, which can bundle to form microbial cellulose ribbons. See, e.g., Ross et al., Microbiol. Mol. Biol. Rev., 55(1):35-58 (1991); Nishi et al., J. Material Sci., 25(6):2997-3001 (1990). Any appropriate method can be used to obtain microbially-derived cellulose. For example, cellulose films can be produced through static incubation of Acetobacter xylinum in a nutrient medium for several days (e.g., 5 to 15 days) at an appropriate temperature (e.g., about 30-37° C.) until films (e.g., 3-dimensional films) are produced. See, e.g., Klechkovskaya et al., Crystallography Reports, 48(5):813-820 (2003). Such films can be about 2-10 mm thick. In some cases, thicker films (e.g., 3-dimensional films having a thickness of 12, 15, 20, 25, 30, or more mm) can be produced using air-lift reactors where air or oxygen is supplied from the bottom of the incubation vessel. With such a configuration, oxygen availability is not confined to the nutrient medium-air interface at the surface of the vessel. In such cases, cellulose layers thicker than 25 mm can be produced.

In some cases, cellulose used as described herein (e.g., cellulose produced by a microorganism) can have a molecular weight between about 1,000 Da and 10,000,000 Da (e.g., between about 1,000 Da and about 5,000,000 Da, between about 1,000 Da and about 2,500,000 Da, between about 1,000 Da and about 1,000,000 Da, between about 1,000 Da and about 500,000 Da, between about 2,000 Da and about 10,000,000 Da, between about 5,000 Da and about 10,000,000 Da, between about 10,000 Da and about 10,000,000 Da, or between about 20,000 Da and about 5,000,000 Da), can have a degree of crystallinity between about 40 percent and 99 percent (e.g., between about 50 percent and 99 percent, between about 70 percent and 99 percent, between about 40 percent and 95 percent, between about 40 percent and 90 percent, between about 40 percent and 80 percent, between about 40 percent and 75 percent, or between about 50 percent and 95 percent), can have a crystal size between about 25 nm and 2 mm (e.g., between about 25 nm and 1.5 mm, between about 25 nm and 1 mm, between about 25 nm and 0.5 mm, between about 50 nm and 2 mm, between about 100 nm and 2 mm, or between about 100 nm and 1 mm), and/or can have a porosity with an average pore size between about 10 nm and 100 μm (e.g., between about 10 nm and 90 μm, between about 10 nm and 50 μm, between about 10 nm and 25 μm, between about 20 nm and 100 μm, between about 50 nm and 100 μm, or between about 25 nm and 10 μm).

In some cases, a degradable biomolecule composition provided herein can be designed to include one or more polypeptides (e.g., structural polypeptides). Examples of polypeptides that can be used to make a degradable biomolecule composition provided herein include, without limitation, collagen (e.g., Type II, Type III, and Type IV collagen), keratin, elastin, fibrin, proteoglycans (e.g., aggregan, versican, decorin, biglycan, fibromodulin, or lumican), or combinations thereof. In general, polypeptides that can be used to make a degradable biomolecule composition provided herein can be obtained by expression of a recombinant nucleic acid encoding the polypeptide or by chemical synthesis (e.g., by solid-phase synthesis or other methods well known in the art, including synthesis with an ABI peptide synthesizer; Applied Biosystems, Foster City, Calif.). In some cases, expression vectors that encode a polypeptide that can be used to make a degradable biomolecule composition provided herein can be used to produce a polypeptide. For example, standard recombinant technology using expression vectors encoding a polypeptide can be used. Expression systems that can be used for small or large-scale production of the polypeptides provided herein include, without limitation, microorganisms such as bacteria transformed with recombinant bacteriophage DNA, plasmid DNA, or cosmid DNA expression vectors containing a nucleic acid sequence that encodes a polypeptide of interest. In general, the resulting polypeptides can be purified according to any appropriate protein purification method. In some cases, a degradable biomolecule composition provided herein can be designed to include one or more recombinant polypeptides.

In some cases, a degradable biomolecule composition provided herein can be designed to include Type I collagen. Type I collagen can be isolated and purified from Type I collagen-rich tissues such as skin, tendon, ligament, and bone of humans and animals as described elsewhere (see, e.g., Miller et al., Methods Enzymol., 82:33-64 (1982) and U.S. Pat. No. 6,090,996).

In some cases, a degradable biomolecule composition provided herein can be designed to include synthetic analogs of polypeptides obtained by genetic engineering techniques. For example, Vitrogen bovine dermal collagen (Cohesion Technologies, Palo Alto, Calif.) can be used. In some cases, a degradable biomolecule composition provided herein can be designed to include genetically engineered collagens such as those marketed by Fibrogen (South San Francisco, Calif.).

As described herein, a degradable biomolecule composition provided herein can be designed to include one or more biomolecule degrading enzymes. The term “biomolecule degrading enzyme” as used herein refers to an enzyme of a degradable biomolecule composition that has enzymatic activity to partially or completely degrade at least one type of biomolecule present in the degradable biomolecule composition. The term “degrade” or “degradation” in context with a biomolecule degrading enzyme refers to a process where an enzyme of a degradable biomolecule composition partially or completely breaks down (e.g., degrades) the biomolecules of a degradable biomolecule composition. The term “biodegrade” or “biodegradation” as used herein refers to a process where a composition is broken down by one or more enzymes produced by a living organism. With respect to the degradable biomolecule compositions provided herein, biodegradation can occur but is not necessarily required. For example, a degradable biomolecule composition can be a wound dressing having cellulose, collagen, cellulose, and possibly a collagenase. When contacted to human tissue, the cellulose can be degraded by the cellulase of the degradable biomolecule composition, and the collagen can be degraded by collagenases incorporated into the material during its production or can be biodegraded by collagenases produced endogenously by the human.

In general, a degradable biomolecule composition provided herein can be designed to include one or more biomolecule degrading enzymes that can partially or completely degrade at least one type of biomolecule to yield a bioabsorbable product (e.g., glucose). As used herein, the term “bioabsorption” refers to a composition that can be absorbed by a tissue or organ of an organism. The degradable biomolecule compositions provided herein can be bioabsorbable and/or biodegradable. For example, a bioabsorbable and/or biodegradable degradable biomolecule composition can be designed such that the composition is easily bioabsorbed and/or biodegraded by human tissue. For example, a degradable biomolecule composition can be a wound dressing having cellulose and cellulase. When contacted to human tissue, the cellulose can be degraded by the cellulase of the degradable biomolecule composition to yield glucose, which is bioabsorbed by the target tissue. In another example, a bioabsorbable and/or biodegradable degradable biomolecule composition can be designed such that the composition is easily bioabsorbed and/or biodegraded by ubiquitous environmental microorganisms or other living organisms after biomolecule degrading enzymes have partially degraded the biomolecules of the composition. For example, a degradable biomolecule composition having cellulose and a cellulose degrading enzyme such as cellulase can be used as an agricultural material. When placed in the soil, the cellulase of the composition can partially degrade the cellulose, and microorganisms present in the soil can biodegrade the partially degraded cellulose to yield glucose, which can be bioabsorbed by living organisms.

Examples of biomolecule degrading enzymes that can be used to make a degradable biomolecule composition provided herein include, without limitation, proteases, cellulases, keratinase, elastinase, chitinase, collagenases, amylases, and combinations thereof. As described herein, a degradable biomolecule composition having one or more biomolecules can be designed to include one or more biomolecule degrading enzymes that have the ability to degrade those one or more biomolecules present within the composition. For example, when a composition is designed to include a polysaccharide biomolecule such as cellulose, the composition can include one or more glycoside hydrolases. In another example, when a composition is designed to include a polypeptide biomolecule such as collagen, the composition can include one or more proteases capable of degrading collagen (e.g., trypsin or collagenase).

In general, when the degradable biomolecule in the degradable biomolecule composition is a polysaccharide (e.g., cellulose or chitin), a biomolecule degrading enzyme can be an enzyme capable of degrading that polysaccharide such as cellulase, amylase, glycogenase, or chitinase. Examples of cellulose degrading enzymes that can be used to make a degradable biomolecule composition provided herein include, without limitation, acid cellulases, hybrid cellulases, neutral cellulases, alkaline cellulases, and combinations thereof. In some cases, cellulose degrading enzymes derived from bacteria, fungi, and protozoans (e.g., endoglucanases, exoglucanases, and β-glucosidases) can be used to make a degradable biomolecule composition provided herein. Examples of such cellulose degrading enzymes include, without limitation, 1,4-β-D-glucan-4-glucanohydrolases; 1,4-β-D-glucan glucanohydrolases; 1,4-β-D-glucan cellobiohydrolases; and β-glucoside glucohydrolases. In some cases, a degradable biomolecule composition provided herein can be designed to include more than one cellulase. For example, a combination of one or more endoglucanases, one or more exoglucanases, and one or more β-glucosidase can promote the complete degradation of cellulose into the bioabsorbable compound glucose.

In general, when the degradable biomolecule in the degradable biomolecule composition is a polypeptide (e.g., collagen, keratin, elastin, or fibrin), a biomolecule degrading enzyme can be a protease enzyme capable of degrading that polypeptide. A degradable biomolecule composition provided herein can be designed to include one or more non-specific proteolytic enzymes such as trypsin and pepsin. In some cases, a degradable biomolecule composition provided herein can be designed to include one or more biomolecule degrading enzymes specific for a particular polypeptide. For example, if polypeptides such as keratin, collagen, or elastin are present in a degradable biomolecule composition provided herein, then one or more biomolecule degrading enzymes such as keratinases, collagenases, or elastinases can be incorporated into a degradable biomolecule composition.

Any appropriate method can be used to obtain biomolecule degrading enzymes for inclusion in a degradable biomolecule composition provided herein. For example, biomolecule degrading enzymes can be obtained commercially, isolated from any of various species that produce the enzyme of interest, or can be produced synthetically using chemical and/or recombinant molecular techniques. In some cases, extracellular cellulase enzymes (e.g., endoglucanases, exoglucanases, and β-glucosidases) produced by aerobic bacteria can be recovered and included in a degradable biomolecule composition provided herein. For example, cellulases can be obtained from, for example, Trichoderma viride, Aspergillus niger, Bacillus subtilis, or Trichoderma reesei.

In some cases, the degradable biomolecule compositions can be engineered to remain stable prior to use. For example, a degradable biomolecule composition provided herein can include one or more biomolecule degrading enzyme inhibitors having the ability to inhibit the degradation of biomolecules within the composition by the biomolecule degrading enzymes present in the composition. In some cases, one or more components of a degradable biomolecule composition provided herein (e.g., biomolecules or biomolecule degrading enzymes) can be lyophilized to reduce or prevent the degradation of biomolecules within the composition by the biomolecule degrading enzymes present in the composition. In some cases, a degradable biomolecule composition can be designed such that the biomolecules and biomolecule degrading enzymes are separated (until use) such that the biomolecule degrading enzymes present in the composition does not degrade the biomolecules present in the composition.

As described herein, degradable biomolecule compositions can be engineered to remain stable using one or more biomolecule degrading enzyme inhibitors. Such biomolecule degrading enzyme inhibitors can be included to prevent premature degradation of biomolecules (e.g., to increase shelf-life of a degradable biomolecule composition). When a degradable biomolecule composition is designed to include a polypeptide biomolecule (e.g., keratin, collagen, or elastin), a biomolecule degrading enzyme can be a protease (e.g., keratinase, collagenase, or elastinase) and a biomolecule degrading enzyme inhibitor can be a generic protease inhibitor or an inhibitor of a specific protease. Examples of biomolecule degrading enzyme inhibitors include, without limitation, carboxyl protease inhibitors (e.g., pepstatin), matrix metalloproteinase inhibitors, and protease inhibitors developed for healthcare applications such as saquinavir, ritonavir, indinavir, nelfinavir, amprenavir, lopinavir, atazanavir, fosamprenavir, tipranavir, and darunavir.

When a degradable biomolecule composition is designed to include a polysaccharide biomolecule such as cellulose, a biomolecule degrading enzyme can be a cellulase and a biomolecule degrading enzyme inhibitor can be a cellulase inhibitor. A cellulase inhibitor can be specific to cellulases, i.e., they do not inhibit or change the action of many molecules other than cellulases. Examples of specific cellulase inhibitors include, without limitation, cellobioimidazole (CBI), fluoro-methyl glucose (FMG), fluoro-methyl cellobiose (FMCB), and 4-O-beta-cellobiosyl-DNJ, 4-O-beta-D-glucopyranosyl-DNJ, and 6-O-beta-cellobiosyl-DNJ. See, for example, York et al., Biochim. Biophys. Acta., 1696(2):223-33. (2004); Bell et al., Botanical Gazette, 143-148 (1960); and Kawaguchi et al., Biosci. Biotechnol. Biochem., 60(2):344-6 (1996)). See also U.S. Patent App. No. 2008/0107619.

In some cases, enzyme inhibitors can be used to deactivate a specific enzyme completely. However, in many cases, an enzyme inhibitor can be used to reduce the activity of the enzyme. For example, 4-O-beta-D-glucopyranosyl-DNJ and 6-O-beta-cellobiosyl-DNJ can be used to partially inhibit the activity of specific cellulases as described elsewhere (Kawaguchi et al., Biosci. Biotechnol. Biochem., 60(2):344-6 (1996)). Incorporating such cellulase inhibitors into a composition provided herein can allow the degradation time of the biomolecule composition to be extended. In some cases, the inhibitor itself can be deactivated over time through the use of another enzyme that degrades the inhibitor. For example, the 4-O-beta-cellobiosyl-DNJ, 4-O-beta-D-glucopyranosyl-DNJ, or 6-O-beta-cellobiosyl-DNJ inhibitors can be degraded using a glycoside hydrolase that cleaves the 1-4 glucan linkage present in the structure of these inhibitors. As the inhibitors are degraded, the activity of the cellulases can be increased.

As described herein, a degradable biomolecule composition can be engineered to remain stable using lyophilization. For example, one or more components of a degradable biomolecule composition provided herein can be lyophilized. In some cases, one or more biomolecules of the degradable biomolecule composition, one or more biomolecule degrading enzymes of the degradable biomolecule composition, or one or more biomolecules and one or more biomolecule degrading enzymes of the degradable biomolecule composition can be lyophilized to reduce or prevent the degradation of biomolecules within the composition by the biomolecule degrading enzymes present in the composition. In some cases, all the biomolecules and biomolecule degrading enzymes of the degradable biomolecule composition can be lyophilized. Lyophilization can be a freeze-drying process that dehydrates the biomolecules and/or biomolecule degrading enzymes while maintaining structural integrity. For example, cellulose films containing cellulase enzymes can retain structural integrity upon lyophilization and rehydration, and the lyophilized enzymes can be biologically active upon rehydration.

To permit degradation of the biomolecules of a degradable biomolecule composition that contains one or more lyophilized biomolecules and/or one or more lyophilized biomolecule degrading enzymes, a solution (e.g., water, saline, a buffered solution, or blood) can be used. In general, rehydration of lyophilized components of a degradable biomolecule composition can return the one or more lyophilized biomolecules present in the composition to their original structure and can restore enzymatic activity of the one or more lyophilized biomolecule degrading enzymes present in the composition. Lyophilized biomolecules and biomolecule degrading enzymes can be rehydrated using any type of aqueous solution such that the composition is activated. In such cases, the biomolecule degrading enzymes of the rehydrated composition can begin to degrade the biomolecules as the biomolecule degrading enzymes regain enzymatic activity and start to degrade their target biomolecules. In some cases, a composition can be formulated for use with a living mammal and can be presented as a lyophilized composition for rehydration in vivo (i.e., when contacted to a target tissue or wound) or ex vivo (i.e., prior to contacting tissue).

In a particular example of lyophilization, the hydrated material can be frozen at a temperature of or below −20° C. to ensure water within material is completely converted into ice. The frozen material can be inserted into a container or flask that is connected to a vacuum chamber where the connection is regulated by a valve. The vacuum pressure can be ˜1 mBar or lower, preferably ˜0.1 mBar or lower. The valve can be opened, and the container or flask can be evacuated to the chamber pressure. At this pressure, the water sublimes but does not melt, i.e., it converts from the solid state directly to the gaseous state without melting. This removes the water from the material and preserves the hydrated structure of the material.

Drying a cellulose material either at room temperature for extended periods or at elevated temperatures in an oven can lead to the collapse of the structure of the cellulose material. When dried, the cellulose can form a dense material which cannot be rehydrated. Moreover, the dried cellulose can form a rigid, relatively inflexible material that easily cracks and is broken into pieces when handled, making storage, shipping, handling, and processing impractical or impossible. This sharp change in material properties of the dehydrated cellulose arises from the creation of extensive hydrogen bonding between fibers of cellulose which collapse together when the water is removed during the drying process.

Lyophilization prevents the collapse of the material that occurs during thermal drying and prevents the formation of hydrogen bonds that cause the material to become rigid and brittle. Lyophilized compositions including cellulose can be porous, flexible, and stable and can exhibit desired mechanical properties for storage, handling, shipping, and for use such as, for example, in would care or tissue scaffold applications. An example of a method of lyophilizing cellulose that can be used to make a composition provided herein is described elsewhere (e.g., U.S. Pat. No. 2,444,124).

Biomolecule degrading enzymes included in a degradable biomolecule composition provided herein also can be lyophilized. Upon rehydration, the lyophilized enzymes can have activity to degrade a specific substrate similar or identical to the activity of the enzymes prior to lyophilization.

Lyophilized compositions and devices provided herein can be rehydrated just before or during use. For example, a lyophilized composition and device can be brought into contact with an aqueous solution or water prior to being placed into contact with biological cells or tissue. Thus, optionally, lyophilized compositions and devices can be rehydrated by contacting the composition and/or device with an aqueous solution or water outside the body of a patient to be treated. In some cases, rehydration of a composition provided herein can be achieved easily and quickly because of the porous structure left after the ice has sublimed during lyophilization.

Thus, in one embodiment, one or more biomolecules and one or more biomolecule degrading enzymes of a degradable biomolecule composition provided herein can be rehydratable. A rehydratable biomolecule can have substantially the same properties in the composition before lyophilization and after rehydration. This means that the composition can be dehydrated to the extent that the degrading enzymes are substantially inactive. These enzymes can be inactive since an aqueous media can be used to both allow the enzyme to exist in a natured state and to allow its transport to the biomolecule surface. In addition, particular enzymes, such as cellulases, may require a water molecule to perform the hydrolysis of the glucan linkage. A rehydratable biomolecule degrading enzyme can have substantially the same properties in a composition and/or device before lyophilization and after rehydration. Thus, for example, after rehydration, the biomolecule degrading enzymes of the composition can be active to degrade the biomolecules of the composition.

In some cases, the biomolecules and biomolecule degrading enzymes can be physically separated. This can be accomplished by joining two or more different materials where at least one material includes one or more biomolecules, and at least one other material includes one or more biomolecule degrading enzymes and a non-biological material or a biomolecule that is not degraded by the biomolecule degrading enzymes. For example, such a compound material can consist of a layer of cellulose material connected to a layer of polyester material where the layer of polyester material contains one or more cellulases. All materials herein can be lyophilized one or more times to preserve the structure and the activity of the enzymes before rehydration.

Combinations of different biomolecules can be included in a degradable biomolecule composition or device provided herein. For example, a degradable biomolecule composition provided herein can be designed to include polysaccharides and polypeptides. Similarly, combinations of different biomolecule degrading enzymes can be included in a degradable biomolecule composition or device provided herein. For example, a degradable biomolecule composition provided herein can be designed to include cellulases and proteases.

Illustratively, a degradable biomolecule composition provided herein can include both cellulose and starch and one or more cellulase and amylase enzymes. Such a composition is useful for controlling the rate of degradation, porosity of the composition over time under the influence of the enzyme system, or chemistry of the material over time under the influence of the enzyme system where as one of the polysaccharides degrades the physical or chemical properties of the remaining polysaccharide will dominate. Compositions can have a combination of one or more structural polysaccharides and/or structural proteins such as, for example, a combination of cellulose and collagen and one or more cellulases and collagenases.

The degradable biomolecule compositions and devices provided herein can optionally include one or more auxiliary agents. Examples of auxiliary agents include, without limitation, cells, biocides, pH-level controlling agents, growth-promoting agents, and pharmaceuticals. For example, a degradable biomolecule composition can be designed to include cells, a biocide agent, and a pH-level controlling agent.

As described herein, a degradable biomolecule composition provided herein can be designed to include cells. Any type of cell can be incorporated into a degradable biomolecule composition provided herein including, without limitation, undifferentiated cells (e.g., stem cells such as mesenchymal stem cells) and differentiated cell types (e.g., osteoblasts, osteogenic cells, osteocytes, osteoclasts, chondroblasts, fibroblasts, macrophages, adipocytes, neurons, cardiomyocytes, and smooth muscle cells). Examples of stem cells that can be included in a degradable biomolecule composition provided herein include, without limitation, stem cells derived from skin, bone, muscle, bone marrow, synovium, or adipose tissue. In some cases, a degradable biomolecule composition provided herein can be designed to include autologous cells. In other cases, a degradable biomolecule composition provided herein can be designed to include cells derived from an animal of the same species (e.g., for an allograft) or from an animal of a different species (e.g., for a xenograft). Any appropriate method can be used to isolate and collect cells. Isolated cells can be rinsed in a buffered solution (e.g., phosphate buffered saline) and resuspended in a cell culture medium. Standard cell culture methods can be used to culture and expand the population of cells. Once obtained, the cells can be contacted with a degradable biomolecule composition provided herein to seed the composition.

A biocide is a chemical substance capable of killing living organisms. Biocides are commonly used in medicine, agriculture, forestry, and in industry. Examples of biocides include, without limitation, pesticides such as fungicides, herbicides, insecticides, algicides, molluscicides, miticides, and rodenticides, and antimicrobials such as germicides, antibiotics, antibacterials, antivirals, antifungals, antiprotozoals, and antiparasites.

In some cases, the degradable biomolecule compositions and devices provided herein can be used as wound dressings or tissue scaffolds. The presence of, for example, glucose in the wound area can present a natural nutrient for the culturing of microbes such as bacteria and fungi. To prevent infection of the wound area due to the presence of increased concentrations of, for example, glucose, the use of a biocide (e.g., an antiseptic compound) can be particularly useful. Thus, one or more antiseptic compounds can be included for an antimicrobial effect to reduce the possibility of infection, sepsis, or putrefaction. Examples of antiseptics that can be included in a composition provided herein include, without limitation, quaternary ammonium compounds, biguanidine derivatives such as polyhexamethylene biguanide (PHMB), octenidine, and sodium hypochlorite.

In some cases, a degradable biomolecule composition provided herein can be designed to include a pH level controlling agent such as a buffering agent. A pH-level controlling agent can be a chemical that controls the pH-level in the environment by, for example, changing the pH of a wound area when a composition is used as a wound dressing and applied to the target tissue. Examples of agents that can be used to control pH include, without limitation, hydrochloric acid, sodium acetate, phosphoric acid, acetic acid, citric acid, lactic acid (e.g., to lower the pH to <7.0), and sodium bicarbonate (e.g., to increase the pH to >7.0). In one embodiment, a pH-level controlling agent is included that keeps a wound environment at pH 6.5 or below, a pH range at which acidic enzymes are most active. In some cases, a pH level controlling agent can be selected according to the desired pH of the composition and/or the desired level of biomolecule degrading enzyme activity. For example, cellulases can be generally divided into four basic groups according to the pH required for optimum enzymatic activity. The optimum pH for acid cellulases can vary between about 4.5 and about 5.0. Hybrid cellulases can have an optimum pH range of about 4.5 to about 7.0. Neutral cellulases can be active from a pH range of about 6.0 to about 8.0, but the optimum pH is about 6.2. The alkaline cellulases can have an optimum pH range from about 7.2 to about 8.5. Alkaline cellulases purified from the fungus Chrysosporium lucknowense can be capable of degrading cellulose at pH values of about 8 to about 12. See, for example, U.S. Pat. No. 5,811,381. In some cases, a degradable biomolecule composition provided herein can be designed to have a pH that falls inside or outside the optimum pH range for the one or more of the biomolecule degrading enzymes present in the composition.

In some cases, a degradable biomolecule composition provided herein can be designed to include one or more growth promoting agents. The term “growth promoting agent” as used herein refers to substances that enhance the healing of tissue and/or organs of an organism. Examples of such substances that can be included in a degradable biomolecule composition provided herein include, without limitation, hormones, cytokines, growth factors, and vitamins such as vitamin A derivatives and vitamin D analogues.

In some cases, a degradable biomolecule composition provided herein can be designed to include one or more pharmaceutical agents such as a substance intended for use in the diagnosis, cure, mitigation, treatment, or prevention of a disease. For example, in topical applications of a degradable biomolecule composition or device provided herein, an included pharmaceutical agent can be an emollient, anti-pruritic, antifungal, disinfectant, scabicide, pediculicide, tar product, keratolytic, abrasive, systemic antibiotic, growth factor, topical antibiotic, hormone, desloughing agent, exudate absorbent, fibrinolytic, proteolytic, sunscreen, antiperspirant, and/or corticosteroid.

This document also provides methods of treating a patient by administering a degradable biomolecule composition and/or device (e.g., a medical device) provided herein to the patient. In particular embodiments, a degradable biomolecule composition provided herein can be configured as a medical device, such as a wound dressing or tissue scaffold. In particular embodiments, biomolecules of a degradable biomolecule composition provided herein can form a 2 or 3-dimensional matrix and preferably a porous 2- or 3-dimensional matrix. In this configuration, the composition or device can be used, for example, as an extracellular matrix (“scaffold”) and/or composite graft as a support system to restore, maintain, or improve tissue function or whole organs such as, but not limited to, skin, bone, nerve, cartilage, heart, liver, bladder, or pancreas.

In particular embodiments, a medical device such as, for example, a wound dressing can include a first layer including biomolecules and a second layer adjoining the first layer that includes biomolecule degrading enzymes. The second layer can include or consists of a material such as, for example, polyester, polypropylene, polyvinylchloride, or polyurethane that is preferably not degradable by the biomolecule degrading enzymes. If the medical device includes rehydratable biomolecules in the first layer and rehydratable biomolecule degrading enzymes in the second layer, the enzymes from the second layer will begin to diffuse into the first layer after hydration resulting in the degradation and/or bioabsorption of the medical device. The first layer and/or the second layer may include further compounds such as, for examples, biocides, pharmaceuticals, growth promoting agents, biomolecule degrading enzyme inhibitors, protease inhibitors, pH level controlling agents, and/or any other additives.

In another embodiment, a degradable biomolecule composition provided herein can be a medical device such as a wound dressing including a first layer including biomolecules, a second bioabsorbable layer adjoining the first layer and preferably not including the biomolecules of the first layer, a third layer adjoining the second layer including biomolecule degrading enzymes. The second bioabsorbable layer can include a synthetic bioabsorbable material such as, e.g., poly-L-lactide, poly-DL-lactide, polyglycolide, polydioxanone, glycolic acid, glycolide, lactic acid, and/or poly-lactic glycolic acid. The thickness of the second layer can be about 10 microns to 1 mm and in part engineered to control the rate of layer dissolution from the third layer to the first layer. The third layer can include or consist of a material such as, for example, polyester, polypropylene, polyvinylchloride, or polyurethane that is preferably not degradable by the biomolecule degrading enzymes. After degradation of the second bioabsorbable layer, the enzymes from third layer will begin to diffuse into the first layer resulting in the degradation and/or bioabsorption of the medical device. The first, second, and/or third layer may include further compounds such as, for examples, biocides, pharmaceuticals, growth promoting agents, biomolecule degrading enzyme inhibitors, protease inhibitors, pH level controlling agents, and/or any other additives.

In a particular example, a medical device can be provided in the form of a film or sheet with a thickness that measures from about 1 mm to about 25 mm or more. One layer of the wound dressing device, the “composition layer,” can include biomolecules and biomolecule degrading enzymes and can be in contact with the wound of a subject to be treated. Another layer, the “polymer layer,” can be in contact with the “composition layer” and can include or consist of a flexible polymer material, such as, for example, polyester, polypropylene, polyvinylchloride, or polyurethane. Such a dressing can include one or more adhesives (e.g., such as those described in U.S. Pat. No. 6,177,482) to facilitate the attachment of the dressing to the target surface area. Such a dressing can further include one or more additional layers such as, for example, an exudate absorbing layer between the “composition layer” and the “polymer layer,” such as described in U.S. Application Publication No. 2006/0161089.

In some cases, the degradable biomolecule compositions and devices provided herein can be configured such that the biomolecule degrading enzymes are evenly distributed with respect to the biomolecules in the composition. This distribution can allow for uniform degradation of the biomolecules. For example, substantially uniform distribution of the biomolecule degrading enzymes within a 2 or 3-dimensional matrix of biomolecules can ensure that the 2 or 3-dimensional matrix is uniformly degraded. Even distribution of the biomolecule degrading enzymes with respect to the biomolecules in the composition can be achieved as described herein.

The degradable biomolecule compositions and devices described herein can be used in numerous applications in addition to wound healing and tissue repair. Thus, for example, a degradable biomolecule composition can be included in pharmaceutical preparations, drug or other active agent delivery devices, in non-medical applications including, but not limited to, filter materials, insulating materials, packaging materials, and in agriculture applications (e.g., mulch film and plant pots). As described herein, a degradable biomolecule composition can include cells. In some cases, such cells can serve as food, for example, for humans or other animals. Thus, in particular embodiments, compositions provided herein can be meat substitutes or dietary supplements. In such embodiments, the inclusion of biomolecule degrading enzymes can be optional.

As described herein, a degradable biomolecule composition can be bioabsorbable and/or biodegradable. If used for medical applications, the degradable biomolecule composition can be partially or completely absorbed by the tissues and organs of a living organism. Biodegradation can, but needs not necessarily, occur. In one embodiment, a degradable biomolecule composition as used for medical applications can be at least bioabsorbable. If used, for example, in agriculture, a degradable biomolecule composition provided herein can be partially or completely biodegraded by microorganisms. In some cases, bioabsorbable and/or biodegradable compositions also can be easily further absorbed and/or degraded, or further bioabsorbed and/or biodegraded by ubiquitous environmental microorganisms or living organisms after the biomolecule degrading enzymes have partially or completely degraded the biomolecules.

As described herein, any appropriate method can be used to make a degradable biomolecule composition. In one embodiment, methods of manufacturing a degradable biomolecule composition provided herein can include lyophilizing biomolecules and biomolecule degrading enzymes. For example, a method of manufacturing a degradable biomolecule composition provided herein can include (a) lyophilization of biomolecules, (b) applying biomolecule degrading enzymes to the lyophilized biomolecules, and (c) lyophilization of the biomolecules and biomolecule degrading enzymes.

In step (a), biomolecules, for example in form of a 2 or 3-dimensional matrix as a sheet, film or scaffold, can be frozen at −20° C. or below. Frozen biomolecules can be rapidly placed into flasks that are connected to a vacuum chamber of a freeze-dryer. The freeze drying operation can depend upon the frozen mass and the particular instrument, but a typical timeframe for freeze drying is 24 to 48 hours.

In step (b), biomolecule degrading enzymes can be applied to the frozen biomolecules of (a). For this purpose, the frozen biomolecules of (a) can be autoclaved and then kept inside a clean hood or container. Biomolecule degrading enzymes can be dissolved into sterile water to form an enzyme solution. One or more auxiliary agents, such as biocides, pharmaceuticals, growth promoting agents, biomolecule degrading enzyme inhibitors, protease inhibitors, pH level controlling agents, and/or any other additives can be optionally added to the enzymes dissolved in the solution, or can be subsequently added. In some cases, the concentration of the enzymes can be based on the mass of the biomolecules and can be calculated so that the weight ratio of enzymes to biomolecules is in the range of 1:5 to 1:500, inclusive. In some cases, the weight ratio of enzymes to biomolecules can be in the range of 1:50 to 1:100, inclusive.

In some cases, biomolecules can form a 2 or 3-dimensional matrix such as a film or sheet, and the enzyme solution can be evenly distributed into the matrix material. This can be done in several ways including: (a) submersion of the 2 or 3-dimensional matrix such as a film into an aqueous solution containing the enzymes and allowing it to saturate for 10 minutes to 20 minutes (where the vessel holding the solution does not permit attachment of the enzyme to its surface, for example, polypropylene); (b) introduction of the enzyme solution into the 2- or 3-dimensional matrix with a pipette or a spray apparatus where the solution is allowed to disperse through the matrix for 10 minutes to 20 minutes in an obturator to prevent water on the surface from fast evaporation and allowing uniform distribution of the enzyme throughout the composition; and (c) vapor deposition of the enzyme solution through liquid ultrasonic atomization, liquid source misted chemical deposition, or molecular vapor deposition.

Before the application to the biomolecules, the enzyme solution can be passed through a 0.2 to 0.45 micrometer filter membrane to remove any microbes or larger particles.

In some cases, the enzymes can be suspended into a buffer solution such as dilute hydrochloric acid when applied to the biomolecules. If the degradable biomolecule composition provided herein is to be used for a wound dressing, the pH of the buffer solution can be adjusted to modify the pH of the wound area and biomolecules. Such an adjustment can be used to optimize the activity of the enzymes, i.e., increase activity or decrease activity to alter degradation time. The rate of degradation of the biomolecules can be controlled to allow for specific applications. In a chronic wound care environment, a degradation time of about 1-4 weeks is desirable. Control of the degradation time can be accomplished by adjusting the enzyme:biomolecule concentrations, selection of the particular enzyme or enzyme systems, control over the local pH which impacts enzyme activity, or by including enzyme inhibitors. Accordingly, the compositions provided herein can degrade partly or completely. Complete degradation can be considered to be achieved when approximately 70%-95% of the biomolecule material (e.g., cellulose material) has been converted to smaller units (e.g., glucose or oligosaccharides).

In step (c), the biomolecules and biomolecule degrading enzymes are again lyophilized. The composition produced in (b) can be again freeze-dried according to the same conditions as described in (a). The composition can be stored in a sealed dehydrated sterile package. This composition is then ready for use and can be stored for prolonged periods (1-2 years or more). To use such a composition, the composition can be removed from the sterile package, saturated with sterile water, and applied (for example to a wound surface). Alternatively, the composition can be saturated with a buffer and inoculated with cells (for example, if used as a tissue scaffold material).

In some cases, a degradable biomolecule composition provided herein can be designed into a drug delivery device. Such a drug delivery device can be a tablet, dragée, or capsule and can include pharmaceuticals/drugs that are encompassed by the degradable biomolecule composition. If the composition includes rehydratable biomolecules and rehydratable biomolecule degrading enzymes, the core of the drug delivery device including the composition can be degraded after hydration releasing the pharmaceutical/drug into its surroundings (e.g., the intestine). In some cases, the drug delivery device material may not be degradable and just pass through the body, delivering the drug compounds as it moves through the body.

The invention will be further described in the following examples, which do not limit the scope of the invention described in the claims.

### EXAMPLES

### Example 1

### Production of Microbial Cellulose Films by Acetobacter xylinus

Microbial cellulose is synthesized by Acetobacter xylinus (e.g., ATCC accession number 23769) in a nutrient medium including, for example, yeast extract, peptone, glucose, citric acid, magnesium sulfate, and sodium phosphate dibasic. Many nutrient compositions and acetobacter strains are known in the prior art. After cultivation at 30-32° C. for 12-13 days, an approximately 3-5 millimeter thick gel film is obtained on the surface of the nutrient solution. Microbial cellulose films are separated and washed with a 0.1N sodium hydroxide solution in deionized water at a temperature of 80° C. in a container submerged in a rocking water bath for 1 hour, or until all biomass has been removed. Subsequently, films are washed with deionized water until a neutral pH has been obtained.

### Example 2

### Initial Freeze Drying of Microbial Cellulose Films

Purified microbial films are frozen at −20° C. Frozen films are rapidly placed into flasks connected to a vacuum chamber of a freeze-dryer (e.g., Labcono Freezone®2.5L Freeze-Dry System). Working vacuum pressure is below 0.133 mbar, and working temperature in the vacuum chamber is below −500° C. The length of the freeze drying operation depends upon the frozen mass and the particular instrument, but a typical timeframe for freeze drying is 24 to 48 hours.

### Example 3

### Introducing Cellulose Degrading Enzymes

Dehydrated films obtained in the initial freeze drying process are cut into desired shapes, i.e., rectangular films measuring 1 in². All films are autoclaved and then kept inside a clean hood or container. Cellulose degrading enzymes are dissolved into sterile water. The concentration of the enzymes is based on the mass of the cellulose film and is calculated so that the weight ratio of enzyme to cellulose film is from 1:5 to 1:500, or more generally from 1:50 to 1:100. A 1 mL enzyme solution is evenly distributed onto the surface of 1 in² rectangular dried film saturating the film. To ensure sterility, the enzyme solution is passed through a 0.2 micrometer or 0.45 micrometer filter membrane to remove any microbes. Freeze dried cellulosic films with applied enzyme solution on the surface are left for about 20 minutes in an obturator which is made of a 10 cm-diameter Petri dish to prevent water on the surface from fast evaporation until enzymes can be distributed evenly into films.

The enzymes can also be suspended into a buffer solution when applied to the cellulose material. The pH of the buffer solution can be adjusted to modify the pH of the wound area and cellulose material. This adjustment can be used to optimize the activity of the enzymes, i.e., to increase or decrease enzymatic activity in order to alter degradation time.

### Example 4

### Second Freeze Drying of Cellulose Compositions

Cellulose films containing enzymes, buffer compounds, and optionally an antiseptic compound are frozen at −20° C. The buffer and antiseptic compounds are incorporated into the enzyme solution and introduced into the biomolecule composition with the enzymes. Enzyme-treated frozen cellulose films are then freeze dried as described above. Films are stored in a sealed dehydrated sterile package.

The films are then ready for use and can be stored for prolonged periods (1-2 years or more). To use the films, the films are removed from the sterile package, saturated with sterile water, and applied to the wound surface. Alternatively, they can be saturated with a buffer and inoculated with cells and used as a tissue scaffold material. The rate of degradation of the cellulose film can be controlled to allow for specific applications. In a chronic would care environment, a degradation time of approximately 1-4 weeks is desirable. Control of the degradation time can be accomplished by adjusting the enzyme:cellulose concentrations, selection of the particular enzyme or enzyme systems and control over the local pH which impacts enzyme activity.

### Example 5

### Degradation of Cellulose Compositions

To optimize the enzymatic degradation of the cellulose material, studies of several cellulases and cellulose combinations were performed. Enzyme combinations and concentrations were optimized to allow complete degradation in 1-2 weeks. Nine commercial cellulose or cellooligosaccharide degrading enzymes were studied to determine the following: 1) time to degradation in freeze-dried microbial cellulose material for a given concentration; 2) the effectiveness of the process for introducing the enzyme into the freeze dried cellulose material; 3) whether the enzyme or enzyme system (more than one enzyme) would function after incorporation into the freeze dried material and the subsequent second freeze drying process; and 4) if freeze drying the antiseptic compound, and the combination of the antiseptic compound and enzymes, impacted the function of these elements. Table 1 contains a list of the 9 commercial enzymes studied (labeled A, B, C, D, E, F, G, H, and I as described on the table) and their degrading functionality.

TABLE 1
Characteristics of Enzymes

| Enzyme | Source/number | Activity and Activity Conditions |
|---|---|---|
| A: (powder) | Sigma/C0615 | Cellulase from Trichoderma viride, pH 5.0, 37° C. |
| B: (powder) | Sigma/C8546 | Cellulase from Trichoderma reesei, pH 5.0, 37° C. |
| C: (powder) | Sigma/C1794 | Cellulase from Aspergillus niger, pH 5.0, 37° C. |
| D: (powder) | Fluka/49101 | β-glucosidase from Aspergillus niger, pH 5.0, 55° C. |
| E: (powder) | Fluka/49106 | β-glucosidase from Bacillus subtilis, pH 6.0, 55° C. |
| F: (powder) | Fluka/49291 | β-glucosidase from Trichoderma sp., pH 4.0 at 37° C. |
| G: (liquid) | Sigma/C2730 | Cellulase from Trichoderma reesei |
| H: (liquid) | Sigma/2605 | Cellulase from Aspergillus sp. (Carezyme 1000L) |
| I: (powder) | Specialty | Neutral cellulase-5000, around pH 7.0, 37-40° C. |
| | Enzymes/5000 | |

Optimization of the enzyme mixture was performed using freeze-dried microbial cellulose materials produced by Acetobacter xylinum. Cellulose samples were prepared as follows:

(1) Preparation of Nutrient Medium

One liter of nutrient medium was prepared using the following ingredients: 20.0 g glucose, 5.0 g yeast extract, 5.0 g bacterial peptone, 2.7 g sodium phosphate dibasic, 1.2 g citric acid, and 5.7 g magnesium sulfate. After mixing and autoclaving, nutrient medium was poured into rectangular pans and then placed into a sterile incubator. A 7-to-14-day static cultivation at 30-31° C. produced cellulose film 3 to 5 millimeters thick. Longer culturing periods produced thicker films, ranging from 5 to 10 millimeters or more. Other culturing processes are known which can produce cellulose films >100 millimeters. See, e.g., Hornung et al., Engineering Life Sci., 6(6):546-551 (2007).

(2) Purification of Cellulose Pellicle

Cellulose films were then separated and washed with 0.1 N sodium hydroxide at 80° C. for one hour to remove bacterial cells. Cellulose pellicles were then washed with deionized water until the occurrence of the neutral reaction.

(3) The pellicles were then autoclaved and kept in a sterile container. Cellulose pellicles were then frozen to −20° C. and freeze dried for 24 hours as described above. Several rectangular 1 in² films were cut and trimmed to exhibit the same mass (about 0.05 g). Enzymes were prepared in 10 mL of sterile buffer solution according to weight ratios of enzyme and substrate (1:50-1:100). To prepare powdered enzymes, 20 mg of enzyme were dissolved in 10 mL sterile buffer. To prepare liquid enzymes, 0.3 mL of enzyme were dissolved in 10 mL sterile buffer. In order to simulate an in vitro wound pH environment, three buffer solutions of different pH were used: Na₂HPO₄-citric acid (pH 4.5), Na₂HPO₄-citric acid (pH 6.0), and Tris-HCl (pH 7.4). All reactions of enzymatic degradation were completed in these three buffer solutions. All buffer solutions were autoclaved before using to ensure that they were sterile. Biocompatible buffer solutions include aqueous solutions of hydrochloric acid, sodium acetate, lactic acid or sodium bicarbonate. One milliliter aqueous enzyme solutions were applied to the cellulose film surface and allowed to absorb into 1 in² dry rectangular cellulose films using a pipette. Films with enzyme solutions were kept in an obturator for approximately 20 minutes to prevent evaporation and to allow for uniform distribution of the enzyme throughout the material samples.

Based on previous studies, none of the following enzyme combinations (A+E, B+E, C+E, A+F, B+F, C+F, A+D, B+D, C+D; see Table 1) exhibited higher degradation activity than single cellulase A, B and C. Other than enzyme D, which exhibited very little degradation activity (about 4% glucose yield) below pH 4.5, neither E nor F was shown to liberate glucose at any pH tested (3.4, 4.5, 6.0 and 7.4) as they are suspected to be pure beta-glucosidases. As a result, cellulases A, B, C, G, H, and I were selected in the initial degradation studies. After the second freeze drying process as described above, dry cellulose films with enzyme loading were placed in 10 mL different buffer water. All the reactions were performed in a sterile incubator at 37° C. for a minimum of 7 days.

### Example 6

### Cellulase Enzyme Absorption into Lyophilized Cellulose Samples

The weights of small cellulose films were measured before and after the enzymes and buffer components were introduced into the cellulose pieces. Ideal absorbed weights for enzymes were 2 mg for powdered enzymes and 36 mg for liquid enzyme G. As enzyme solutions were prepared by buffer solution, control trials can be used to eliminate the impact of buffer absorption. Experiments were repeated three times. Table 2 details the weight variance of samples and the buffer solution in which the enzymes were suspended. The estimated ability of lyophilized cellulose samples to absorb enzyme was calculated using the following formula: m=(m₂− m̅₁̅)/3, where m₁ is a control absorbed weight after freeze-drying small cellulose pieces, and m₂ is an enzyme absorbed weight after freeze-drying cellulose pieces. It should be noted that these values also contain the weight of buffer compounds.

These results indicate that both powdered enzymes and liquid enzymes are readily able to be introduced into cellulose films. As shown in Table 2, the cellulase films have the highest ability to absorb enzymes at pH 6.0 relative to absorption at different pH values.

TABLE 2
Estimated Ability Of Enzyme Absorbed Into Cellulose Films.

| | pH 4.5 | | | | | | | pH 6.0 | | | | | | |
|---|---|---|---|---|---|---|---|---|---|---|---|---|---|---|
| | (buffer 1 citric acid-sodium | | | | | | | (buffer 2 citric acid-sodium | | | | | | |
| | phosphate dibasic) | | | | | | | phosphate dibasic) | | | | | | |
| Weight | | m2 | | | | | | | m2 | | | | | |
| (mg) | m1 | A | B | C | G | H | I | m1 | A | B | C | G | H | I |
| 1 | 23.7 | 25.7 | 27.0 | 26.2 | 39.0 | 33.6 | 25.8 | 23.2 | 29.6 | 27.3 | 27.0 | 43.9 | 33.9 | 26.2 |
| 2 | 22.8 | 26.1 | 25.4 | 26.5 | 39.4 | 34.9 | 23.1 | 23.5 | 28.8 | 25.6 | 28.0 | 45.4 | 34.3 | 26.9 |
| 3 | 23.3 | 25.1 | 25.1 | 26.3 | 36.5 | 30.6 | 24.3 | 23.5 | 27.6 | 27.1 | 29.0 | 46.1 | 29.2 | 27.6 |
| m | | 2.3 | 2.5 | 3.0 | 15.0 | 9.7 | 1.1 | | 5.3 | 3.3 | 4.6 | 21.7 | 9.1 | 3.5 |

| | pH 7.4 | |
|---|---|---|
| | (buffer 3 Tris-HCl) | |
| Weight | | m2 |

| (mg) | m1 | A | B | C | G | H | I |
|---|---|---|---|---|---|---|---|
| 1 | 7.6 | 9.5 | 9.9 | 10.5 | 24.3 | 14.6 | 8.7 |
| 2 | 7.8 | 9.3 | 8.7 | 9.9 | 24.7 | 15.2 | 10.1 |
| 3 | 8.3 | 10.2 | 9.3 | 9.7 | 24.8 | 15.5 | 10.3 |
| m | | 1.8 | 1.4 | 2.1 | 16.7 | 7.2 | 1.8 |

### Example 7

### Degradation of Lyophilized Cellulose-Cellulase Enzyme Composites

Physical examination of 18 cellulose samples containing enzymes A-I at 3 different pH values was performed each day for seven days. Degradation was scored by assigning one of the following scores: None, Slight, Moderate, Extensive, Nearly Complete, and Complete. “None” indicates that no degradation was observed. “Slight” indicates that some particles were observed and/or the composite had a faint milky appearance. “Moderate” means that many particles were observed and/or the composite had a milky appearance; “Extensive” means complete fragmentation and/or milky appearance; “Nearly Complete” means few particles were observed and the solution was almost clear. “Complete” degradation means the solution was virtually clear.

As shown in Table 3, cellulases A, B, C, and G exhibited better degradation ability at or below pH 6.0. After 7 days, cellulases A, B, C, and G almost degraded cellulose completely to glucose. Cellulases A, B, C, and G exhibited little degradation ability at a pH of 7.4. By increasing the enzyme concentration of cellulases A, B, C, and G by a factor of 3 at a pH of 7.4, the cellulose was almost completely degraded after another 7 days. Cellulases H and I did not exhibited good degradation ability at any selected pH. A pH 6.0, however, cellulase I displays slight degradation on the second day, which is quicker than at pH 7.4.

TABLE 3
Observed Degradation Degrees

| | | 1st | | 3rd | | 5th | | |
|---|---|---|---|---|---|---|---|---|
| | | day | 2nd day | day | 4th day | day | 6th day | 7th day |
| pH 4.5 | A | M | E | NC | NC | NC | NC | NC |
| | B | M | E | NC | NC | NC | NC | NC |
| | C | S | M | E | NC | NC | NC | NC |
| | G | S | M | NC | NC | NC | NC | NC |
| | H | N | N | N | N | N | N | N |
| | I | N | N | N | N | S | N | N |
| pH 6.0 | A | S | M | E | NC | NC | NC | NC |
| | B | S | M | E | NC | NC | NC | NC |
| | C | S | S | M | NC | NC | NC | NC |
| | G | S | M | M | NC | NC | NC | NC |
| | H | N | N | N | N | N | N | N |
| | I | N | S | S | S | S | S | S |
| pH 7.4 | A | N | N | N | S | S | S | S |
| | B | N | N | N | S | S | S | S |
| | C | N | N | N | S | S | S | S |
| | G | N | N | N | S | S | S | S |
| | H | N | N | N | N | N | N | N |
| | I | N | N | N | N | S | S | S |

### Example 8

### Efficiency of Converting Cellulose to Glucose Using Cellulases

The efficiency of converting cellulose to glucose for the enzymes examined above was assessed using high performance liquid chromatography (HPLC), which is able to quantify the concentration of glucose in the solution left behind. Samples of the solutions that contained the cellulose sample and the cellulose degrading enzymes were collected on the 1st day, 4th day and 7th day to compare the glucose yield using HPLC. The values presented in Table 4 were calculated in the form of the ratio of the percent of actual glucose yield to maximum possible glucose yield: Ratio percent=(actual glucose yield/maximal ideal glucose yield)×100. Maximal ideal glucose yield=(m₁×180)/162.

The data in Table 4 demonstrates that enzymes A and C behaved as acid cellulases and were able to degrade over 87% and 85% of the cellulose to glucose, respectively, at a pH of 4.5. The conversion efficiency only dropped to 78% and 82% when the pH is changed to 6.0. Thus, either of enzymes A and C can be used for degrading cellulose in wounds where the pH is within this range either naturally or when subjected to a biological buffer solution where the pH is kept in this range.

TABLE 4
Glucose Yield Data.

| | pH 4.5 | | | | | | pH 6.0 | | | | | |
|---|---|---|---|---|---|---|---|---|---|---|---|---|
| | Buffer 1: citric acid-sodium | | | | | | Buffer 2: citric acid-sodium | | | | | |
| | phosphate dibasic | | | | | | phosphate dibasic | | | | | |
| (%) | A | B | C | G | H | I | A | B | C | G | H | I |
| 1st day | 57.15 | 34.19 | 43.94 | 39.72 | 0.00 | 21.79 | 33.46 | 27.33 | 29.96 | 28.35 | 0.00 | 24.09 |
| 4th day | 74.31 | 54.38 | 74.63 | 62.98 | 0.00 | 23.82 | 62.56 | 29.50 | 45.79 | 40.97 | 0.00 | 25.99 |
| 7th day | 87.43 | 68.78 | 85.92 | 80.57 | 19.42 | 26.49 | 78.79 | 52.64 | 82.28 | 66.55 | 18.49 | 28.89 |

pH 7.4
Buffer 3: Tris-HCl

| (%) | A | B | C | G | H | I |
|---|---|---|---|---|---|---|
| 1st day | 23.21 | 22.36 | 22.00 | 24.64 | 0.00 | 0.00 |
| 4th day | 22.82 | 22.27 | 21.82 | 25.18 | 0.00 | 0.00 |
| 7th day | 23.95 | 23.00 | 23.57 | 26.43 | 0.00 | 0.00 |

### Example 9

### Bacterial Growth During Degradation of the Composition

If the composition is used for a wound dressing, the presence of glucose in the wound area presents a natural nutrient for the culturing of microbes such as bacteria and fungi. To prevent infection of the wound area due to the presence of increased concentrations of glucose, the use of an antiseptic compound was explored. Two test materials (A and B) were prepared. Material A is a lyophilized cellulose film with cellulase C, and material B is a lyophilized cellulose film with cellulase C plus antiseptic Polyhexamethylene Biguanide (PHMB). Media and inoculation conditions are shown in Table 5. Lyophilized cellulose samples A and B were incubated under conditions 1 and 2 for one month. Using media of tryptic soy agar, the samples were put in the solution to perform enzymatic degradation. On the 2nd, 8th, and 14th day, the degrading solution was taken out and plated on the agar media. The agar media plates were incubated for one month according to condition 3.

TABLE 5
Media and Inoculation Condition.

| Conditions | Media | Incubation temperature |
|---|---|---|
| 1 | Tryptic soy broth (30 g/L) | 36-37° C. |
| 2 | Tryptic soy broth (30 g/L) | 25-26° C. |
| 3 | Tryptic soy agar medium (40 g/L) | 25-26° C. |

As shown in Table 6, one of the “A” test materials without PHMB exhibited microbial growth, whereas no microbial growth was observed from any sample tested that incorporated PHMB. Moreover, no observable reduction in cellulase enzyme activity was found when PHMB was present.

TABLE 6
Microorganism Growth on Composites.

| | | Microbial growth (after 1 month | | |
|---|---|---|---|---|
| Test materials | Condition | for conditions 1 and 2) | | |
| A (no PHMB) | 1 | − | | |
| | 2 | + | | |
| | 3 | 2^{nd} day | 8^{th} day | 14^{th} day |
| | | − | − | − |
| B (with PHMB) | 1 | − | | |
| | 2 | − | | |
| | 3 | 2^{nd} day | 8^{th} day | 14^{th} day |
| | | − | − | − |

### Example 10

### Medical Devices Including Cellulose, Cellulose Degrading Enzymes, and an Antiseptic Agent

Microbial cellulose is synthesized by Acetobacter xylinum. After a 5 to 15 day cultivation at 30-37° C., an about 2-10 millimeter thick cellulose film is obtained on the surface of the nutrient solution. Microbial cellulose films are separated and washed with a 0.1N sodium hydroxide solution in deionized water at a temperature of 80° C. in a container submerged in a rocking water bath for 1 hour or until all biomass has been removed. Subsequently, films are washed with 0.5% acetic acid and then with distilled water until the desired pH has been obtained. For chronic wound care applications, the pH is expected to be in the range of 5.5-7.5. See Schneider et al., Arch. of Dermatological Res., 278:413-420 (2007). The material is then autoclaved to ensure sterility as well known in the art. Purified microbial films are frozen at −20° C. Frozen films are then rapidly placed into flasks that are connected to a vacuum chamber of the freeze-dryer as described above. The freeze drying operation depends upon the frozen mass and the particular instrument but a typical timeframe for freeze drying is 20 to 24 hours. Dried films obtained via first freeze drying are cut into desired shapes, i.e., rectangular films measuring 1 in². Cellulose degrading enzymes are prepared into enzyme solution with sterile water and PHMB (0.15%). The weight ratio of enzyme and cellulose film was approximately 1:50. One milliliter of enzyme solution is evenly distributed onto the surface of 1 small rectangular dried film, saturating the film. To ensure sterility, the enzyme solution is passed through a 0.2 micrometer or 0.45 micrometer filter membrane to remove any microbes. Freeze dried films with enzyme solution applied to the surface are placed in an obturator for approximately 20 minutes, or until enzymes can be distributed evenly into films, to prevent water on the surface from fast evaporation. Cellulose films are then frozen again at −20° C. Enzyme treated frozen cellulose films are then lyophilized as described above. Films are stored in a sealed sterile package. These films are then ready for use and can be stored for prolonged periods (months to years). To use the films, the films are removed from the sterile package, saturated with sterile water, and applied to the wound surface. Alternatively, they can be inoculated with cells and used as a tissue scaffold material.

### Example 11

### Medical Devices Including Cellulose, Cellulose Degrading Enzymes Having Different pH Dependencies, and an Antiseptic Agent

Microbial cellulose films are produced by culturing Acetobacter Xylinum bacteria as described herein, and the lyophilization step of microbial cellulose film is conducted as described herein. Cellulose degrading enzymes and an antiseptic is introduced as described in Example 10 except the enzyme solution contains a combination of alkaline, hybrid, neutral and/or acetic enzymes, in particular, an endoglucanase and a 13-glucosidase from each of these 3 classes of enzymes. For example, a combination of cellulase A or C in addition to cellulases derived from the fungus Chrysosporium lucknowense. The second lyophilization step of the cellulose material is conducted as described in Example 10.

### Example 12

### Cellulose Wound Care or Tissue Scaffold Medical Device Including Cellulose Degrading Enzymes, Compounds to Alter the Wound or Tissue pH, and an Antiseptic Agent

Microbial cellulose films are produced by culturing Acetobacter xylinum bacteria as described herein, and the lyophilization step of microbial cellulose film is conducted as described herein. Cellulose degrading enzymes and an antiseptic is introduced as described in Example 10 except that cellulose degrading enzymes are prepared into enzyme solution with PHMB (0.15%) and a buffer solution which will exchange, when the cellulose film is rehydrated before use, with the wound solution and shift the pH. The buffer solution consists of the following compounds: water, hydrochloric acid, sodium acetate, and/or lactic acid. The second lyophilization step of cellulose material is conducted as described in Example 10.

### Example 13

### A Multi-Layer Wound Care Medical Device Including a Cellulose Composition with Rehydratable Cellulose Degrading Enzymes

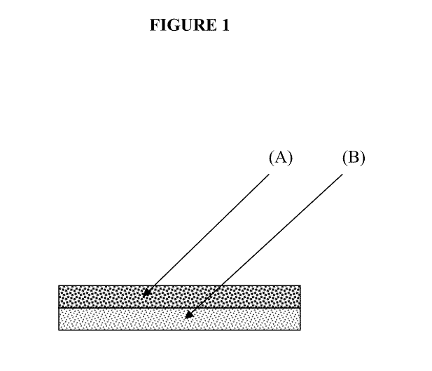

With reference to FIG. 1, a medical device is designed to include a first layer of rehydratable cellulose material (1) optionally further including biocides, pharmaceuticals, growth promoting agents, biomolecule degrading enzyme inhibitors, protease inhibitors, and/or pH level controlling agents as described herein, whose thickness measures about 1 mm to about 10 mm, which is brought into contact with the wound or injured tissue area. The second layer (2) of material is in contact with the first layer and includes polypropylene, polyvinylchloride, or polyurethane, and a quantity of one or more rehydratable cellulose degrading enzymes such as endoglucanases, exoglucanases, and/or 13-glucosidases. The thickness of the second layer is about 1 mm to 25 mm. The second layer optionally including biocides, pharmaceuticals, growth promoting agents, biomolecule degrading enzyme inhibitors, protease inhibitors, and/or pH level controlling agents. Once hydrated, enzymes from the second layer will begin to diffuse into the first layer resulting in the degradation and bioabsorption of the material.

### Example 14

### A Multi-Layer Wound Care Medical Device Including Rehydratable Cellulose Material with Rehydratable Cellulose Degrading Enzymes

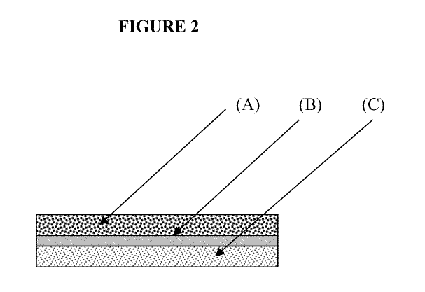

With reference to FIG. 2, a medical device is designed to include a first layer (1) of rehydratable cellulose material optionally further including biocides, pharmaceuticals, growth promoting agents, biomolecule degrading enzyme inhibitors, protease inhibitors, and/or pH level controlling agents as described herein, whose thickness measures about 1 mm to about 10 mm, which is brought into contact with the wound or injured tissue area. A second layer (2) of material is in contact with the first layer (1) and consists of a synthetic bioabsorbable material such as poly-L-lactide, poly-DL-lactide, polyglycolide, polydioxanone, glycolic acid, glycolide, lactic acid, or poly-lactic glycolic acid. The thickness of this layer is about 10 microns to 1 mm and in part engineered to control the rate of layer dissolution when hydrated. The third layer of material (3) is in contact with the second layer (2) and includes polypropylene, polyvinylchloride, or polyurethane, and a quantity of one or more rehydratable cellulose degrading enzymes such as endoglucanases, exoglucanases, and/or β-glucosidases. The thickness of this layer is about 1 mm to 25 mm. This third layer (3) optionally further includes biocides, pharmaceuticals, growth promoting agents, biomolecule degrading enzyme inhibitors, protease inhibitors, and/or pH level controlling agents as described herein. Once hydrated, the second synthetic bioabsorbable material layer will begin to degrade. Once it is no longer continuous, enzymes from third layer will begin to diffuse into the first layer resulting in the degradation and bioabsorption of the material.

Although this example details a two dimensional layered structure, a three dimensional structure can be formed where layer (1) is an outer layer, layer (2) is an intermediate layer, and layer/region (3) is an inner layer or region. The three dimensional structure can be cylindrical, spherical, cubic, or have any arbitrary shape. Such three dimensional devices can be advantageous, for example, for tissue engineering applications where it is desired to have a delay in degradation in the material as the tissue grows on and within the outer layer and then once layer 2 degrades, the enzymes contained in the inner layer/region are allowed to interact with layer 1 degrading the material. Layer/region (3) may be of very small size (<1 to 100 mm³) and contain concentrated enzymes so as to limit the size of the region.

### Example 15

### Applying Degradable Biomolecule Compositions to Mammals

An animal study was performed to evaluate the degradable biomolecule compositions containing enzymes at different concentrations following application on dermo-epidermic wounds in the guinea pig. Twelve animals were treated at two sites for 21 days where the wound was created. Macroscopic evaluations of the wounds were done twice a week until termination. Histological analysis was conducted on treated sites to assess the healing process and safety parameters 21 days after wounding.

A total of twelve young female guinea pigs (strain: Dunkin Hartley; CHARLES RIVERS Laboratories, L'Arbresle, France) were used and weighed approximately between 345 g and 395 g at the beginning of the study. Dermo-epidermic wounds with the size of approximately 4 cm² were surgically created in each guinea pig on day 0. One wound was created on each side of the vertebral column in each guinea pig. Five-group degradable sample patches were applied on the wounds of each designated animal. Group sample #1 was control patches with no enzyme, no buffer dependence but PHMB. Group sample #2 was the patches containing a low concentration of enzyme (5 mg/patch, enzyme C shown in Table 1) at pH 3.5 (buffered with 0.1 M citric acid and 0.1 M sodium citrate) with PHMB (0.15% w/w). Patches in group sample #3 contained a low concentration of enzyme but at pH 4.0 and PHMB. Patches in group sample #4 contained a high concentration of enzyme (15 mg/patch) at pH 4.0 and PHMB as well. Group sample #5-1 included patches with a high concentration of enzyme at pH 3.5 with PHMB and glycerol (5% v/w), and whereas patches in group sample #5-2 contained a same high concentration of enzyme at pH 4.0 with PHMB and glycerol.

All pre-lyophilized sample patches were moistened with sterile saline in a Petri dish prior to application to the wound. The volume of saline used ranged from 0.4 mL to 0.6 mL, and the time of moistening remained between 1 minute and 8 minutes corresponding to different patches.

Once the wound site was created and the patch was applied, each wound site was covered with a non-adhesive interfacial dressing of polyethylene of 2.5 cm² (Buster) and a semi-permeable adhesive polyurethane film (Tegaderm®, 3M, France). Subsequently, an adhesive bandage (sterile gauze and UrgoStrapping®, Urgo, France) was used to secure the dressings over the wound.

The animals were observed daily for general health and wound healing macroscopic evaluation. At termination (Day 21), the animals were terminated to expose the wound site to give a histopathological evaluation.

Compared to control patches, all the degradable patches exhibited different degradability at different pH buffered wound sites. The proposed ranking in terms of degradable performance was, in decreasing order: (most) group 4>group 5-2>group 5-1>group 2>group 3>group 1 (less). Another ranking in terms of the overall histopathological evaluation of wound healing was, in decreasing order: (most) group 4=group 1=group 5-2>group 3=group 5-1>group 2 (less) (Table 7).

TABLE 7
Summary of histopathological evaluation results.

| | |
|---|---|
| Group sample #1: | Wounds showed moderate macrophagic infiltrates associated with |
| no enzyme, buffer, with | multinucleated giant cells and product residues, a slight heterophils |
| PHMB | and lymphocytes within the granulation tissue. The material residue |
| | was observed both at the superficial dermal and lower layers. The |
| | wound were epithelialized with a moderate level of epidermal |
| | differentiation, collagenic maturation and presence of crusts. |
| Group sample #2: | Wounds showed superficial tissue degeneration, necrosis and some |
| low enzyme concentration, | crusts were observed in all sites. Minimal epitheliazation was |
| pH 3.5 buffer, with PHMB | observed. The granulation tissue was slightly more inflammatory |
| | than the control group. A few material debril were observed. |
| Group sample #3: | Wounds exhibited very slight better healing features (less signs of |
| low enzyme concentration, | inflammation) than group 2 despite the fact that there was more |
| pH 4.0 buffer, with PHMB | residual material within the granulation tissue. |
| Group sample #4: | Wounds showed an improved healing performance compared to |
| high enzyme concentration, | group 3. Moderate signs of inflammation associated with less |
| pH 4.0 buffer, with PHMB | material debris than group 3 were observed. |
| Group sample #5-1: | Wounds showed that inconsistency was observed among the sites |
| high enzyme concentration, | with slight to moderate signs of inflammation associated with a |
| pH 3.5 buffer, with PHMB | few material debris. |
| and glycerol | |
| Group sample #5-2: | Wounds showed an improved healing performance compared to |
| high enzyme concentration, | group 5-1. This group appeared slightly less inflammatory |
| pH 4.0 buffer, with PHMB | compared to group 5-1. |
| and glycerol | |

These results indicate that on dermo-epidermic wounds, the use of the degradable biomolecule compositions containing a high enzyme concentration at pH 4.0 buffered with PHMB was able to acquire the effectively degradable outcome and relatively excellent histopathological regeneration of wounds. These results also demonstrate that there is no negative effect associated with the inclusion of tested enzymes in the material. Based on the results of these tests, additional compositions can be designed to have improved performance characteristics. In addition, all materials containing enzymes demonstrated improved degradability, indicating that these materials have use in other applications including tissue engineering, drug delivery, and other medical, agricultural, scientific, and food uses.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered composition comprising one or more biomolecules and one or more biomolecule degrading enzymes.

2. The composition of claim 1, wherein said one or more biomolecules are rehydratable biomolecules.

3. The composition of claim 1, wherein said one or more biomolecule degrading enzymes are rehydratable biomolecule degrading enzymes.

4. The composition of claim 1, wherein said composition is bioabsorbable.

5. The composition of claim 1, wherein said composition is biodegradable.

6. The composition of claim 1, wherein said one or more biomolecules are selected from the group consisting of keratin, collagen, elastin, starch, cellulose, chitosan, and chitin.

7. The composition of claim 1, wherein said one or more biomolecule degrading enzymes are capable of degrading said one or more biomolecules.

8. The composition of claim 1, wherein said one or more biomolecules form a 2- or 3-dimensional matrix.

9. The composition of claim 8, wherein said matrix is porous.

10. The composition of claim 1, wherein said composition comprises cellulose and one or more cellulose degrading enzymes.

11. The composition of claim 10, wherein said cellulose degrading enzymes are selected from the group consisting of endoglucanases, exoglucanases, and β-glucosidases.

12. The composition of claim 10, wherein said cellulose degrading enzymes are selected from the group consisting of acid cellulases, hybrid cellulases, neutral cellulases, and alkaline cellulases.

13. The composition of claim 1, wherein said composition further comprises at least one compound selected from the group consisting of biocides, pharmaceuticals, growth promoting agents, biomolecule degrading enzyme inhibitors, protease inhibitors, and pH level controlling agents.

14. The composition of claim 1, wherein said one or more biomolecules are lyophilized biomolecules.

15. The composition of claim 1, wherein said one or more biomolecule degrading enzymes are lyophilized biomolecule degrading enzymes.

16. The composition of claim 1, wherein said one or more biomolecules and said one or more biomolecule degrading enzymes are made rehydratable by a lyophilization process.

17. The composition of claim 1, wherein said composition comprises one or more chemicals capable of adjusting the pH of an environment contacted with said composition.

18. The composition of claim 17, wherein said one or more chemicals are selected from the group consisting of hydrochloric acid, sodium acetate, phosphoric acid, acetic acid, citric acid, lactic acid, and sodium bicarbonate.

19. The composition of claim 1, wherein said one or more biomolecules are structural proteins, and said one or more biomolecule degrading enzymes are structural protein degrading enzymes.

20. The composition of claim 1, wherein said composition further comprises polyhexamethylene biguanide.
